(19) United States
(12) Patent Application Publication
(10) Pub. No.: US 2008/0318883 A1
(43) Pub. Date: Dec. 25, 2008

(54) Anti-aging supplement

(75) Inventors: Takefumi Hatanaka, Tokyo (JP); Hirofumi Hatanaka, Tokyo (JP)

Correspondence Address: NIXON & VANDERHYE, PC, 901 NORTH GLEBE ROAD, 11TH FLOOR, ARLINGTON, VA, 22203, US

(73) Assignee: Elson Corporation, Tokyo (JP)

(21) Appl. No.: 11/812,455

(22) Filed: Jun. 19, 2007

(51) Int. Cl. A61K 31/7052; A61P 43/00

(52) U.S. Cl. 514/44

(57) ABSTRACT

An anti-aging supplement is disclosed having effective amounts of β1,3D glucan, deoxyribonucleic acid, and body's hyaluronan-yielding row material composed of at least one amino sugar selected from a group consisting of N-acetylglucosamine and glucosamine salts.

### BACKGROUND OF THE INVENTION

1. Technical Field of the Invention

The present invention relates to anti-aging supplements and, more particularly, to an anti-aging supplement for enabling a body age to be improved.

2. Description of the Related Art

In recent years, research and development work has been undertaken to achieve anti-aging. It has been turned out aging factors are caused by drops in reactive oxygen scavenging ability and immune strength due to increasing age and stress, a degradation in metabolism of cells due to increasing age and decrease in mucopolysaccharide, yielding moisturizing raw material of skins, and raw material yielding cartilage. Among these aging factors, stress seems to be mostly related to the progress of aging for the reason described below. That is, when encountered with a stress, a large amount of reactive oxygen is generated inside the living body. This results in a difficulty for the living body to adequately scavenge reactive oxygen generated in the living body. Therefore, such an increase in the amount of reactive oxygen causes damage to deoxyribonucleic acid (DNA) of the living body, resulting in an increase in the degree of aging.

Deoxyribonucleic acid (DNA) repeatedly plays a role as an element to create new cells and DNA is produced in a lever of the living body. However, DNA production ability progressively decreases with the progress of age after an elapse of a period of active growth. This results in not only aging and slowdown in cell cleavage but also the incidence of incomplete cells, promoting the degree of aging.

Further, DNA is damaged due to reactive oxygen as mentioned above, resulting in a difficulty of creasing normal cell cleavage of cells. As DNA is damaged due to the factors such as the reactive oxygen, deterioration occurs in the metabolism, causing incomplete DNA to be progressively copied during the cell cleavage. Thus, ribonucleic acid continuously creates incomplete proteins due to command of the damaged DNA, resulting in a progress of aging in the cells. Thus, decline of DNA yielding ability and damage to DNA constitute serious factors of aging phenomenon.

Thus, due to various factors mentioned above, especially, a body age glows older than an actual age. In modern measuring technologies on metabolism, there has been a success of a body scanner, available in a market, which can precisely measure such a body age. Therefore, it becomes possible to check a progress of aging based on the body age measured by the body scanner. On the other hand, there is another way to check the progress of aging in terms of a serum homocysteine.

In the related art, an attempt has heretofore been made to retard aging by dosing the living body with an anti-aging composition, composed of hyaluronic acid and carnitine, for thereby retarding the aging of the skins as disclosed in U.S. Pat. No. 6,585,987.

Further, another attempt has heretofore been made to dose the living body with vitamin-B complex composed of folic acid, vitamin B12 and vitamin B6, etc., to lower a body level of a serum homocysteine for thereby achieving antiaging as disclosed Japanese Patent Application Publication No. 2003-155234.

Our research and development work has revealed that the greater the metabolism, the lower will be the body age regardless of the actual age in some extent. Thus, it has been turned out that improving the metabolism of the living body enables a recovery of the body age in an efficient manner.

None of the related art anti-aging foods suggests how to improve the metabolism of the living body for thereby effectively lowering the body age to get young.

### SUMMARY OF THE INVENTION

The present invention has been completed with a view to addressing the above issues and has an object to provide an anti-aging supplement that is safe and effective in increasing a metabolism while lowering a body age for thereby effectively achieving an anti-aging effect.

To achieve the above object, a first aspect of the present invention provides an anti-aging supplement comprising effective amounts of β1,3D glucan, deoxyribonucleic acid, and body's hyaluronan-yielding row material composed of at least one amino sugar selected from a group consisting of N-acetylglucosamine and glucosamine salts.

The use of β1,3D glucan is important in scavenging reactive oxygen generated in the living body to prevent damage to DNA for thereby improving a metabolism. Further, β1,3D glucan remarkably stimulates macrophages and natural killer (NK) cells in immune system cells for thereby extremely improving immune function to remove serious factor causing a progress of aging. Moreover, β1,3D glucan, when combined with DNA, further speeds up the metabolism and facilitate the metabolism to convert body's hyaluronan-yielding row material to hyaluronic acid in the living body for thereby lowering a body age in an effective manner. Body's hyaluronan-yielding row material is effective for forming chondroitin sulfate and hyaluronic acid in the living body and has biologic effects as described below, thereby contributing to the anti-aging effect.

Another aspect of the present invention provides an anti-aging supplement comprising effective amounts of a) β1,3D glucan present in an amount of from about 10.0% to about 40.0% by weight based on the total weight of the anti-aging supplement, b) from about 10.0% to about 40.0% by weight of deoxyribonucleic acid based on the total weight of the anti-aging supplement, and c) from about 25.0% to about 45.0% by weight of at least one amino sugar, selected from a group consisting of N-acetylglucosamine and glucosamine salts, based on the total weight of the anti-aging supplement.

### DETAILED DESCRIPTION OF PREFERRED EMBODIMENTS

Now, an anti-aging supplement of an embodiment according to the present invention will be described below in detail. However, the present invention is construed not to be limited to such an embodiment described below and technical concepts of the present invention may be implemented in combination with other known technologies or the other technology having functions equivalent to such known technologies.

The anti-aging supplement comprises effective amounts of β1,3D glucan, deoxyribonucleic acid, and hyaluronan-yielding row material composed of at least one amino sugar selected from a group consisting of N-acetylglucosamine and glucosamine salts.

β1,3D glucan includes at least one of soluble and non-soluble polysaccharides, composed of glucose units, which include, for instance, polysaccharides in 1,3-glycosidic bond, 1,4-glycosidic bond or 1,6-glycosidic bond. Typical examples of β1,3D glucan may include β1,3D glucan derived from yeasts, barley, oat, aureobasidium and mushrooms.

In particular, β1,3D glucan is important in effectively exhibiting a strong scavenging function to eliminate reactive oxygen resulting from external factors such as psychologic stress or solar ultraviolet radiation, while stimulating hematopoietic stem cell so as to increase fresh blood. Further, β1,3D glucan, when combined with DNA, remarkably speeds up the metabolism of a living body and facilitate the metabolism to convert body's hyaluronan-yielding row material to hyaluronic acid in the living body for thereby achieving anti-aging effect in an effective manner.

Further, as used herein, the term “DNA” refers to deoxyribonucleic acid that includes nucleoprotein extracted from fish's spermary. This DNA is composed of a principal component of nucleotide. Necessary nucleotide is synthesized in the living body and deoxyribonucleic acid in food is decomposed into nucleoside, after which nucleoside is absorbed in the intestine of the living body for reuse in synthesizing deoxyribonucleic acid in the living body. This provides physiologic effects as described below.

That is, nucleotide and nucleoside forming the component elements of deoxyribonucleic acid have physiologic effects as listed below including:

(1) Metabolism-improving effect and nutritional improvement such as fat-metabolism improvement;

(2) Immune-function boosting effect such as an increased power of resistance to the attack of cancer or other illness, antiaging effect and an effect of boosting thymus lymphoid cells;

(3) Effect of boosting and regenerating a liver energy metabolism effective in regeneration of a liver, the suppression of decomposition of protein, an energy-metabolism improvement for liver ischemia, and recovery of the liver from damage thereto;

(4) Effect of conserving intestine including an effect of protecting mucosa of small intestine and recovering the intestine after diarrhea;

(5) Skin protecting action including a moisturizing action, a UV-absorption blocking action and activator action on the skin; and

(6) Effect of suppressing a drop in a liver function due to the aging.

It has been turned out that with β1,3D glucan combined with nucleotide and nucleoside of deoxyribonucleic acid, the living body can have remarkably increased scavenging effect due to synergetic effect for thereby suppressing damage to DNA to a minimal limit. Under such a remarkably increased scavenging effect, dosing deoxyribonucleic acid (DNA) from the outside addresses a drop in DNA-synthesizing capability in the living body decayed with aging. This promotes normal cell division of the living body. This suppresses DNA from being damaged due to reactive oxygen, while making it possible to promote body's biologic metabolism such as intestine cells, bone-marrow cells, hairy root cells and genital cells. This is quite effective in retarding the aging, while activating biologic metabolism of skin cells for thereby achieving skin rejuvenation.

Further, β1,3D glucan remarkably stimulates macrophages and natural killer (NK) cells in immune system cells for thereby extremely improving immune function to remove serious factor causing the aging. Thus, a further effective anti-aging effect can be achieved.

Example of glucosamine includes at least one amino sugar selected from a group consisting of N-acetylglucosamine and glucosamine salts. Among these, N-acetylglucosamine may be preferably used as the most effective component. N-acetylglucosamine functions as an important component of a natural moisturizing factor (NMF) in performing a moisture regulating function especially in the skin cells. Moreover, this component is a substance to become a raw material (hereinafter referred to as “body's hyaluronan-yielding row material”) for forming chondroitin sulfate and hyaluronic acid in the living body and has biologic effects as described below.

That is, (1) an effect of forming organizations such as bones, cartilages, skins, fingernails and hair.

(2) An effect of promoting the formation of cartilages.

(3) Action of boosting a joint function and alleviating pain.

(4) An effect of promoting the synthesis of collagen.

(5) An effect of creating beautiful skin and alleviating the aging of skin.

N-acetylglucosamine is present everywhere in the living body and declination occurs in ability of forming N-acetylglucosamine with a progress of aging. As a result, a remarkable reduction occurs in a rate of production of N-acetylglucosamine in the living body. This causes a decrease to occur in a moisturizing factor of skin with the resultant occurrence of dry skin with an increase in mottled and wrinkled skin. Therefore, with N-acetylglucosamine combined with vitamin group and β1,3D glucan in dosing, these combined components provide a synergetic effect, enabling a remarkable recovery of the biologic effect decayed with the aging.

Amino sugar may be preferably combined with mucopolysaccharides.

Examples of mucopolysaccharides include low molecular hyaluronic acid and chondroitin. Example of chondroitin may preferably include chondroitin sulfate.

Further, the anti-aging supplement of the present embodiment may further preferably include ascorbic acid and B complex vitamins.

Example of ascorbic acid may preferably include ascorbate such as ascorbyl palmitate and calcium ascorbate. Vitamin C, also known as ascorbic acid, is necessary for the synthesis of collagen and is used as an antioxidant. Vitamin C fights infection, reduces inflammation, heals wounds, reduces the risk of heart disease, lowers cholesterol, reduces the risk of lung, stomach, and esophageal cancers, reduces cervical epithelial abnormalities, inhibits N-nitrosamine, and reduces the severity of colds. Dosing ascorbic acid to a living body avoids ascorbic acid from depleting in surrounding areas in the vicinity of macrophages when bearing a load such as stress, thereby suppressing drops in immune function and reactive-oxygen scavenging ability. This makes it possible to remarkably improve reactive-oxygen scavenging ability when subjected to the load such as stress. This results in a capability of minimizing damage to cell membranes, DNA and enzymes of the living body.

Example of B complex vitamins may preferably include vitamin B1 thiamine, vitamin B2 riboflavin, vitamin B3 niacin, vitamin biotin, vitamin B6 pyridoxine, vitamin folic acid, vitamin B12 cyanocobalamin, and pantothenate.

Vitamin B1 thiamin helps keep collagen-rich connective and mucous membranes healthy, helps to maintain smooth muscles, helps in the formation of blood cells, and is necessary for proper nervous system function.

Vitamin B2 riboflavin is necessary for healthy hair, nails, and mucous membranes and is involved in red blood cell formation, antibody production, and overall growth.

Vitamin B3 niacin helps in the production of most of the sex hormones, dilates blood vessels, lowers cholesterol, and helps maintain blood circulation.

Vitamin biotin is effective in metabolizing fatty acid and amino acids.

Vitamin B6 pyridoxine is involved in the production of ribonucleic acid (RNA) and deoxyribonucleic acid (DNA) and many other reactions in the body.

Vitamin B6, when combined with folic acid and vitamin B12, has been found to decrease homocysteine levels. Vitamin B6, when combined with deoxyribonucleic acid (DNA) dosed to the living body from the outside, is used for speeding up a normal metabolism of cells based on deoxyribonucleic acid (DNA) dosed to the living body from the outside.

Vitamin folic acid is essential in the production of red blood cells, the production of hormones, and the synthesis of DNA.

Vitamin B12 cyanocobalamin is necessary for overall metabolism, the function of the nervous system, metabolism of folic acid, and the production of red blood cells.

Pantothenate is important for the production of adrenal gland hormones, increases overall energy, increases autonomic nervous function, helps convert food into energy, and helps retard aging. Pantothenic acid is necessary for promoting the production of high-density lipoprotein cholesterol (HDL) that lowers low-density lipoprotein-cholesterol (LDL) for thereby minimizing the causes of aging. If an attempt is neglected to lower LDL, then, lipid covering the surface of LDL is bonded with active oxygen. This results in the oxidation of LDL, causing oxidized LDL to adhere onto the blood vessels. This results in an increase the aging.

Although no particular limitation is intended, anti-aging supplement of the present embodiment may take the form of powder, granular particles, capsules and tablets, etc. Also, in an alternative form, anti-aging supplement of the present embodiment may be utilized as raw materials for foods, additive for food and may be incorporated in food products such as cakes, breads and drinks, etc.

The amount of anti-aging supplement of the present invention may be preferably dosed to each adult in an amount ranging from 100 to 3500 mg and, more preferably, a value ranging from 200 to 2500 mg. If the amount of dose per one day is less than 100 mg, then, it takes a long time before we get a real sense of an anti-aging effect, that is, before we can recognize a reduction in a body age. In contrast, if the amount of dose per one day is greater than 3500 mg, then, no further increased anti-aging effect is obtained. It will be appreciated that the amount of anti-aging supplement to be dosed to one adult may suitably determined in such a range on consideration of age and physical condition.

Although the present invention will be described below more in detail with reference to examples, no limitation is intended and the present invention may take various modifications and alternatives.

With Examples described below, the degree of aging is represented with indexes such as a serum homocysteine level and a body age. The serum homocysteine level refers to a “serum homocysteine concentration”. Further, the body age differs from an actual age and refers to an “age inside a body”, that is, an “age of a body” such as an age of a body function including muscles, bones, blood vessels and internal organs, etc. The body age is measured on the ground of an aging tendency based on a basal metabolism of a body composition. The body composition refers to a “composition forming a body” that includes lipids, muscles, bones and water, etc.

### EXAMPLE 1

(Effect of Suppressing Increase in Serum Homocysteine Level)

In conducting tests on anti-aging effect, various kinds of samples were prepared. First samples were prepared each in a dummy capsule filled with 350 mg of starch component. Second samples of the present invention were prepared each in a 350 mg of capsule filled with 50 mg of starch, 90 mg of yeast derived 1,3/1,6D glucan (manufactured and sold by Elson Corporation under the name “Beta King”), 120 mg of N-acetylglucosamine (manufactured and sold by Yaizu Suisan under the name of “marine Sweet”), 90 mg of deoxyribonucleic acid (DNA) (manufactured and sold by Nichiro under the same “DNA-A”). Third samples of the present invention were prepared each in a 350 mg of capsule filled with 50 mg of premixed vitamins containing vitamin A, B-complex vitamins and vitamin C (sold by Nutrition Japan under the name “DSM”), 90 mg of yeast derived 1,3/1,6D glucan (manufactured and sold by Elson Corporation under the name “Beta King”), 120 mg of N-acetylglucosamine (manufactured and sold by Yaizu Suisan under the name of “marine Sweet”), 90 mg of deoxyribonucleic acid (DNA) (manufactured and sold by Nichiro under the same “DNA-A”).

The tests included 12 women in their twenties to fifties, which are divided in three groups. Four dummy capsules (1) and four present capsules (2) and (3) were dosed to the three groups, respectively, every day for four weeks, upon which serum homocysteine levels were evaluated to measure the anti-aging effects.

In measuring the serum homocysteine levels, the serum homocysteine levels of the individuals were measured at fixed times before the start of dosing and after the dosing, with average values being adopted. Also, the serum homocysteine levels before the start of dosing had an average value of 9.7 μmol/L±2.0.

Test results are summarized in Table 1, indicating the serum homocysteine levels after dosing the four capsules to the three groups every day for four weeks, which is listed below. From this Table, remarkable reductions were confirmed in the groups dosed with the capsules (2) and (3) of the present invention. Women of 1st group were dosed with the capsules of the dummy sample (1) for four weeks. Women of 2nd and 3rd groups were dosed with the capsules (2) and (3), respectively, for four weeks.

TABLE 1

| | CAPSULES | AVERAGE VALUE OF SERUM |
|---|---|---|
| GROUPS | TYPES | HOMOCYSTEINE LEVELS (μmol/L) |
| 1ST GROUP | (1) | 9.6 ± 1.8 |
| 2ND GROUP | (2) | 6.8 ± 1.9 |
| 3RD GROUP | (3) | 6.5 ± 1.7 |

As will be apparent from this Table 1, women of the 2nd and 3rd groups had a success in achieving a reduction of the serum homocysteine levels by a value greater than 30%, with the serum homocysteine levels hitting lowered values less than a standard level of 7.0 μmol/L.

(Test on Body Age)

The tests on body ages included 15 women in their thirties to fifties, which are divided in three groups. Four dummy capsules (1) and four capsules (2) and (3) were dosed to the three groups, respectively, every day for four weeks in the same manner as the tests mentioned above, upon which body ages were measured to check the anti-aging effects. The body age was measured using an inner scanner Model No. BC-527-SV (manufactured and sold by Tanita Co. Ltd.

Test results are summarized in Table 2, indicating the body ages after dosing the four capsules to the three groups every day for four weeks, which is listed below. From this Table, remarkable reductions were achieved in the body ages of the groups dosed with the capsules (2) and (3) of the present invention. Women of 1st group were dosed with the capsules of the dummy sample (1) for four weeks. Women of 2nd and 3rd groups were dosed with the capsules (2) and (3), respectively, for four weeks.

TABLE 2

| | | AVERAGE BODY | AVERAGE BODY |
|---|---|---|---|
| | | AGES BEFORE | AGES AFTER |
| GROUP | CAPSULES | TEST | TEST |
| 1ST GROUP | (1) | 48 | 48 |
| 2ND GROUP | (2) | 49 | 44 |
| 3RD GROUP | (3) | 47 | 41 |

From Table 2, it will be clear that the anti-aging supplement of the present invention is quite effective in lowering the body age regardless of the actual age. This seems to be due to the fact that dosing β1,3D glucan in combination with deoxyribonucleic acid and amino sugar, selected from a group consisting of N-acetylglucosamine and glucosamine salts, enables remarkable improvement in metabolism for thereby achieving remarkable reduction in body age. Especially, when combined with multi-vitamins, these components provide further remarkable anti-aging effect as achieved in the third group.

While the specific embodiment of the present invention has been described in detail, the present invention is not limited to the particular embodiment set forth above. It will be appreciated by those skilled in the art that various modifications and alternatives to those details could be developed in light of the overall teachings of the disclosure. For instance, the anti-aging supplement may be dosed not only humans but also animals such as livestock, pets including dog, cats and fishes, etc.

What is claimed is:

1. An anti-aging supplement comprising effective amounts of:
β1,3D glucan;
deoxyribonucleic acid; and
body's hyaluronan-yielding row material composed of at least one amino sugar selected from a group consisting of N-acetylglucosamine and glucosamine salts.

2. The anti-aging supplement according to claim 1, further comprising:
vitamin A, B-complex vitamins and vitamin C.

3. An anti-aging supplement comprising effective amounts of:
a) β1,3D glucan present in an amount of from about 10.0% to about 40.0% by weight based on the total weight of the anti-aging supplement;
b) from about 10.0% to about 40.0% by weight of deoxyribonucleic acid based on the total weight of the anti-aging supplement; and
c) from about 25.0% to about 45.0% by weight of at least one amino sugar, selected from a group consisting of N-acetylglucosamine and glucosamine salts, based on the total weight of the anti-aging supplement.

4. The anti-aging supplement according to claim 3, further comprising:
d) from about 5.0% to about 25.0% of vitamin A, B-complex vitamins and vitamin C.
